(19) United States
(12) Patent Application Publication
(10) Pub. No.: US 2013/0333704 A1
(43) Pub. Date: Dec. 19, 2013

(54) Wrist Worn Portable Breathing Apparatus

(71) Applicant: Jeffery Duncan, Orlando, FL (US)

(72) Inventor: Jeffery Duncan, Orlando, FL (US)

(21) Appl. No.: 13/525,414

(22) Filed: Jun. 18, 2012

(51) Int. Cl. A62B 7/04; A61M 16/04; A61M 16/20; A62B 9/06

(52) U.S. Cl. CPC A62B 7/04; A62B 9/06; A61M 16/0488; A61M 16/208
USPC 128/205.22

(57) ABSTRACT

The present invention, WristOx, is designed as a means of providing a user with means of extracting multiple breaths in situations that do not allow for the use of a traditional SCUBA configuration. The interchangeable compressed air cylinders allow for the user to exchange spent cylinders for full cylinders on the fly. Designed to be worn unobtrusively on the arm, the present invention foregoes the need for hoses or a second stage regulator, allowing for use in activities such as kayaking, spelunking, or even as an additional line of defense for divers and fire rescue personnel.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

Devices for breathing in mediums other than air are essential for applications such as diving and fire rescue. These activities, as well as a variety of others rely heavily on the use of self-contained underwater breathing apparatuses (SCUBA) to provide the participant with a constant, regulated flow of air. The use of SCUBA gear has allowed for completion of previously impossible endeavors and has opened the door for many future applications as well. While the applications and achievements involving SCUBA gear are varied and widespread, there are limitations and drawbacks to the use of a SCUBA system.

SCUBA systems require the use of compressed air. To allow for a reasonable period of use, the compressed air is normally contained in long, cylindrical tanks. These tanks are typically stored on the user's back (up to three tanks at a time). While the tanks allow for a greater flexibility of movement, they are also restrictive in the user's range of motion. Due to the size and heft of the tanks, users cannot run, jump, or swim as effectively as they normally would (traditional SCUBA gear may weigh up to 75 pounds). The loss of normal movement eliminates the possibility of using SCUBA gear in extreme environments such as big wave surfing or avalanche skiing. In extreme activities it is necessary to allow for a full range of motion with no additional added weight, to ensure maneuverability and survivability when unforeseen circumstances arise.

The size of traditional SCUBA tanks also limits the access of the user to various openings. Openings that a person would normally fit through are no longer accessible due to the added dimensions of the tanks situated on the user's back. This loss of access is vitally important during search and rescue operations in collapsed buildings or plane crashes. Without a smaller breathing apparatus, lives of victims may be lost due to inability to access their location.

As one would surmise, for a tank system secured to a user's back, there must be a means with which to transfer the tank contents to the user's nose or mouth for inhalation. As the contents of the cylinders are under pressure (˜3,000 pounds per square inch), the air must flow through a regulator to prevent damage to the user's lungs. In a typical SCUBA configuration, this is accomplished using a two-stage regulator. The first stage attaches to the cylinder and drops the air pressure from ˜3,000 psi to ˜150 psi. The second stage regulator, located at the user's mouth, is connected to the first stage regulator via a hose. The second stage regulator further reduces the air pressure from ˜150 psi to ambient pressure.

While having the first and second stage regulators separated by a hose in a traditional SCUBA configuration is necessary to reduce the necessary lung inhalation exertion while diving, a hose can be an item of concern with other SCUBA applications. While completing search and rescue missions, for example, a hose can easily become ensnared or tangled in debris, causing failure of the system.

Traditional SCUBA tanks are made of aluminum. Aluminum has become an accepted standard thanks to its light weight, cost, and favorable strength characteristics. A typical aluminum scuba tank weighs 30 pounds empty. Filling the tank with 80 cubic feet of air (at 3,000psi) adds seven pounds, bringing the total to 37 pounds. While this weight is not of primary concern while in the water, it becomes paramount if the tank is used in applications requiring physical exertion on dry land. For this reason, more and more applications are transitioning from aluminum tanks to tanks that have been developed using carbon fiber. Carbon fiber wrapped cylinders, often referred to as “Type 3” cylinders have a seamless metal liner over-wound on all surfaces by a composite reinforcement that provides between 75% and 90% of the strength of the vessel. The liner provides the rest of the strength in addition to acting as a rigid membrane for gas containment and impact resistance. Carbon fiber cylinders are the lightest weight cylinders available today and have a safety factor far exceeding that of steel or aluminum. Carbon fiber wrapped tanks have an allowable pressure of more than 4,500 pounds per square inch, nearly 50% more than an aluminum tank of similar proportions, at a third the weight.

While various temporary breathing solutions have been patented (U.S. Pat. No. 7,156,092, U.S. Pat. No. 4,996,982), there is nothing available for users that can be unobtrusively worn and allows for quick exchange of compressed air cylinders. Presently available items are either too awkward to be worn while participating in extreme activities or are not able to be “re-filled on-the-fly.” It is the intent of the present invention to provide a means for solving both concerns practically and efficiently.

### BRIEF SUMMARY OF THE INVENTION

The present invention provides a highly portable breathing apparatus that can be used in applications in which previous self-contained breathing apparatuses were not an option. WristOx is an emergency air supply and breathing apparatus that can be worn on the body so as to not impede the user's motion or reaction to environmental conditions. The dimensions of the unit are such that it can be strapped to a user's forearm, upper arm, thigh or lower leg and provide needed emergency breathing capability without interfering with the bending, flexing or rotating of the user's arms or legs. In addition, the weight of the unit is less than two pounds, having minimal effect on the overall strength and abilities of the user.

The present invention consists of multiple interchangeable cylinders connected to a housing, which contains a single stage regulator, a pressure gauge and a mouthpiece. The housing is attached to a strap, which can be securely attached to the user's body or to an article of clothing.

Because WristOx has no exposed hoses or cabling, there is no fear of being ensnared by any environmental hazards (low hanging limbs, outcroppings of rocks, or any of the debris one would encounter in a wreckage). The carbon wrapped cylinders are connected directly to the housing, which is directly connected to the mouthpiece. For those users seeking an even cleaner solution, an optional cover can be installed over the cylinders, creating a smooth housing with no exposed surfaces.

As the present invention is intended primarily for near surface use (1 Atmosphere of pressure or less), a single stage regulator can be used, reducing the complexity of the invention and further reducing the size of the present invention. The use of a single stage regulator has no effect on the cylinder capacity, mitigating any concerns of useable air volume.

Unlike prior art U.S. Pat. No. 7,156,092, the present invention has the capability to house multiple air tanks. While the apparatus is not intended for extended periods of use, WristOx does have the capability of removing empty cylinders and replacing with spare full cylinders at any time or location, without the need for an air compressor or any specialized equipment.

Unlike prior art U.S. Pat. No. 4,996,982, the present invention can be attached to the body, much like a wristwatch. This allows the user the ability to constantly monitor the whereabouts and functionality of the unit without the fear of dropping the emergency breathing apparatus. It is also important to note that the present invention is capable of storing air at a greater pressure, thus allowing for more air to be stored in a smaller package.

Unlike prior art Design Pat. No. 351,207, the present invention houses at least two interchangeable carbon fiber wrapped cylinders containing compressed air at a minimum pressure of 4500 psi at capacity.

The present invention has a variety of applications, some of which include: big wave surfing, avalanche skiing, whitewater rafting/kayaking, spear fishing, snorkeling, exiting a plane crash, high altitude activity, exiting a building fire, spelunking, and swimming in rip currents. In addition, a quantity of WristOx devices can be stored near fire extinguishers, etc. to be distributed in the event of emergency in buildings or larger vehicles (ships, buses, airplanes, etc).

This device may also potentially be used in medical applications for respiratory delivery requiring a mix of other gases under pressure.

### BRIEF DESCRIPTION OF THE SEVERAL VIEWS OF THE DRAWINGS

A preferred embodiment of the present invention is illustrated in and by the following drawings in which like reference numerals indicate like parts and in which:

FIG. 1 is a front perspective view of the fully assembled unit. The unit is shown with two cylinders attached to the housing.

FIG. 2A is a front perspective view of the full unit in an exploded view.

FIG. 2B is a front view of the full unit in an exploded view.

FIG. 3 shows multiple views of the regulator housing for a unit having two cylinders.

FIG. 4 illustrates top, side and perspective views of a single cylinder.

FIG. 5A is a hidden line perspective view of the poppet and lever assembly (3).

FIG. 5B is a perspective view of the poppet and lever assembly (3).

FIG. 6 illustrates bottom, front, left, and top views of poppet and lever assembly (3).

FIG. 7 provides front, left, bottom, and perspective views of rupture disc plug (4).

FIG. 8 illustrates perspective, front, and side views of rupture disc (5).

FIG. 9 details the front, back, left, and front perspective views of pressure gauge (8).

FIG. 10 illustrates the side, top, and perspective views of check valve (7).

FIG. 11 provides front, top and rear perspective views of purge cap.

FIG. 12 illustrates the front, top, and perspective views of diaphragm (2).

FIG. 13 illustrates the front, top, and perspective views of exhaust disc (10).

FIG. 14 is an itemized assembly parts diagram for the unit.

FIG. 15 is a cross section of the assembled unit.

FIG. 16 is a shaded front view of an exploded and itemized assembly for the unit.

FIG. 17 is a shaded perspective view of an exploded assembly for the unit.

### DETAILED DESCRIPTION OF THE INVENTION

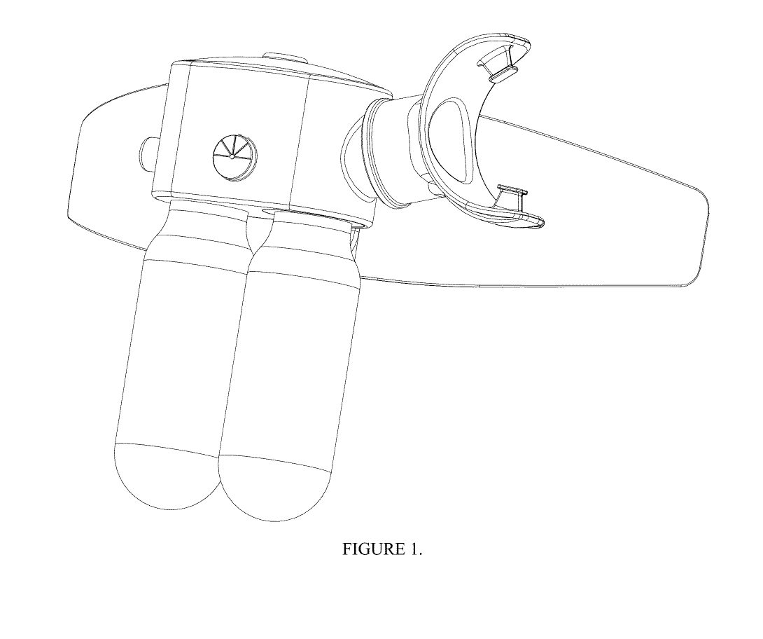

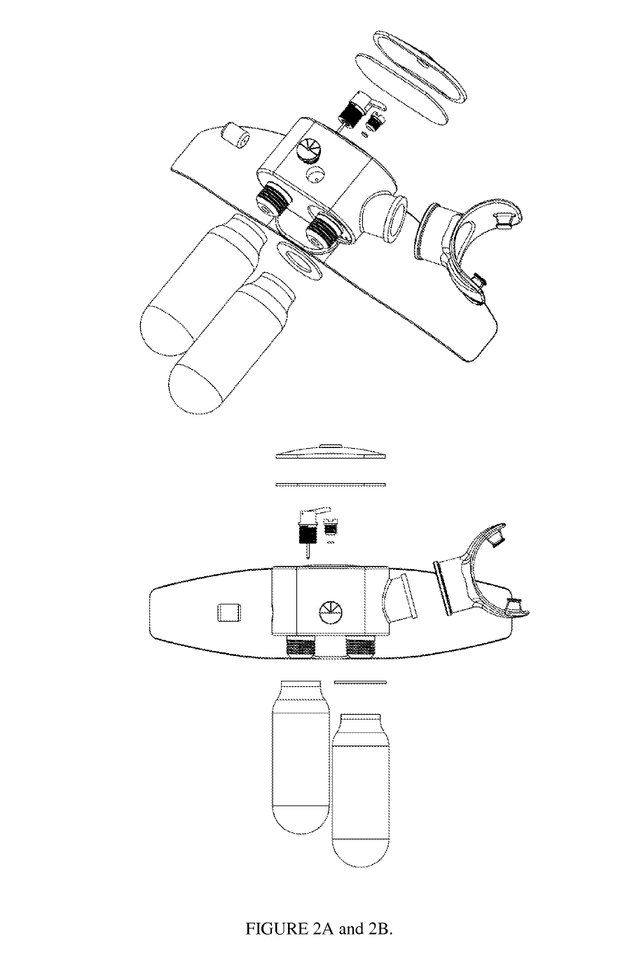

The present invention is designed to be mechanically simple and operator friendly. The WristOx, FIG. 1, is a small, unobtrusive, self-contained breathing apparatus intended to provide a limited air supply during emergency situations. The present invention is mechanically similar to most self-contained breathing apparatuses. The WristOx has pressurized cylinders (9); often referred to as tanks or vessels, that house compressed air. The composition of the air in the cylinders (9) is identical to atmospheric air: the compressed air is approximately 78% nitrogen, 21% oxygen, and 1% trace elements. The pressurized cylinders (9) are Type 3 cylinders with a carbon fiber weave around the perimeter of the vessels. The carbon fiber reinforcement creates the majority of the strength of the vessel, allowing the metal walls to be thinner, ultimately reducing the weight of the cylinders (9). Each cylinder (9) will have an approximate diameter of one and a half inches (1.5″) and an approximate length of five inches (5″). The carbon fiber wrapped cylinders can be pressurized to one and a half times the allowable pressure of an aluminum cylinder, allowing for the compressed air to be stored at 4,500 pounds per square inch. Each cylinder (9) is capable of storing 0.42 cubic feet of air. The preferred embodiment of the present invention has two carbon fiber wrapped cylinders (9), providing the user with 0.84 cubic feet of air.

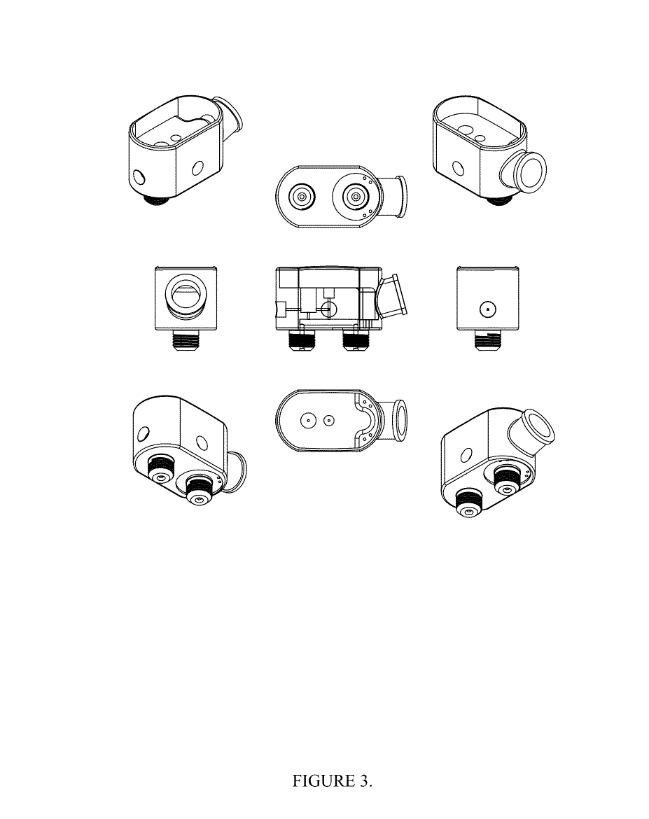

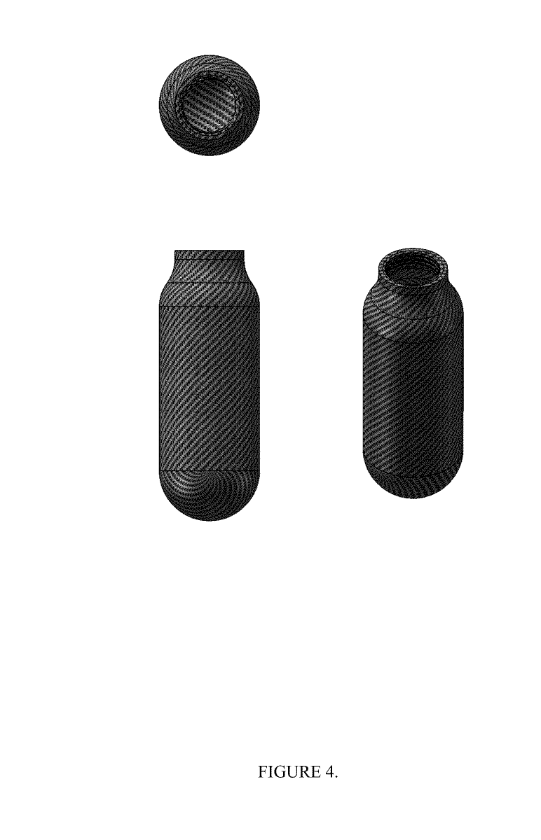

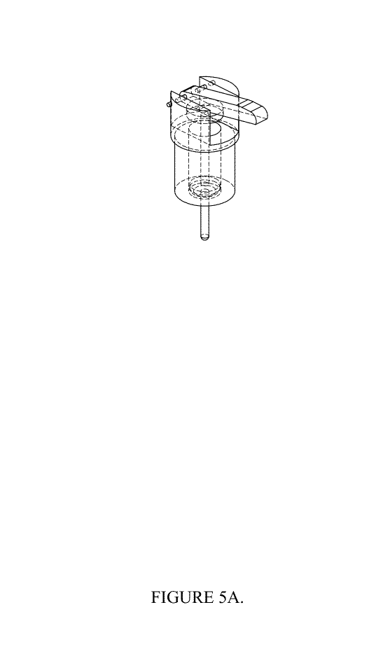

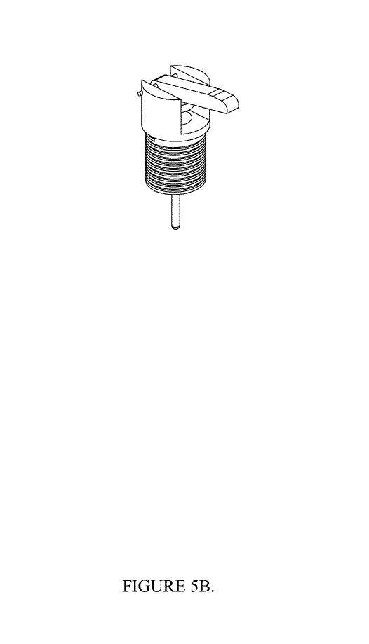

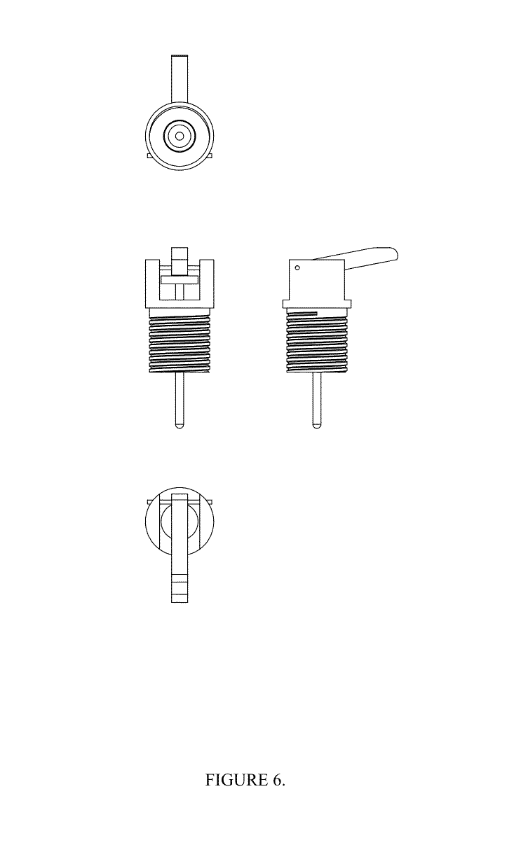

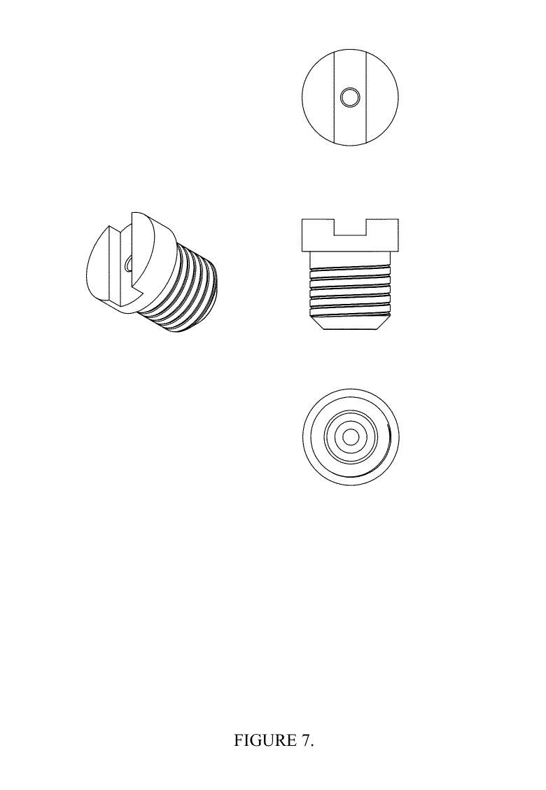

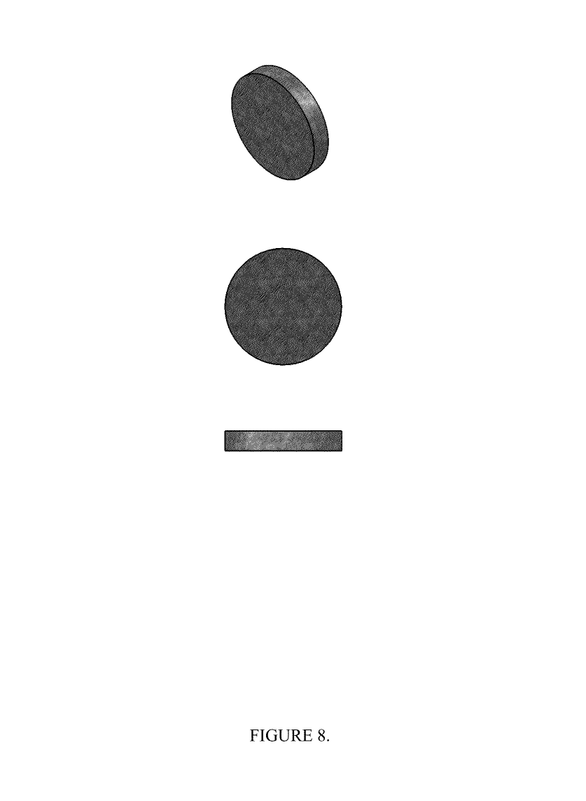

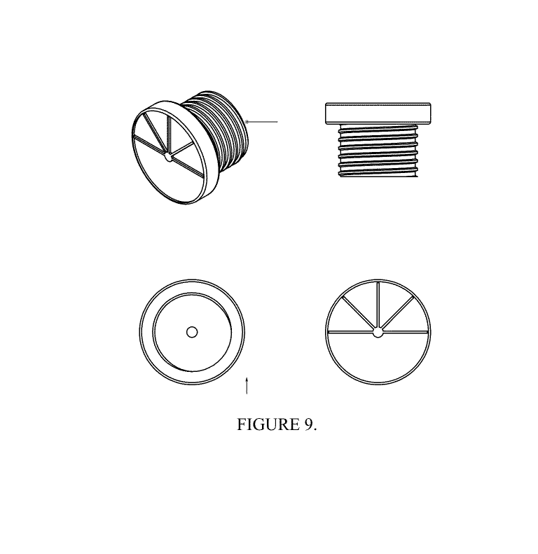

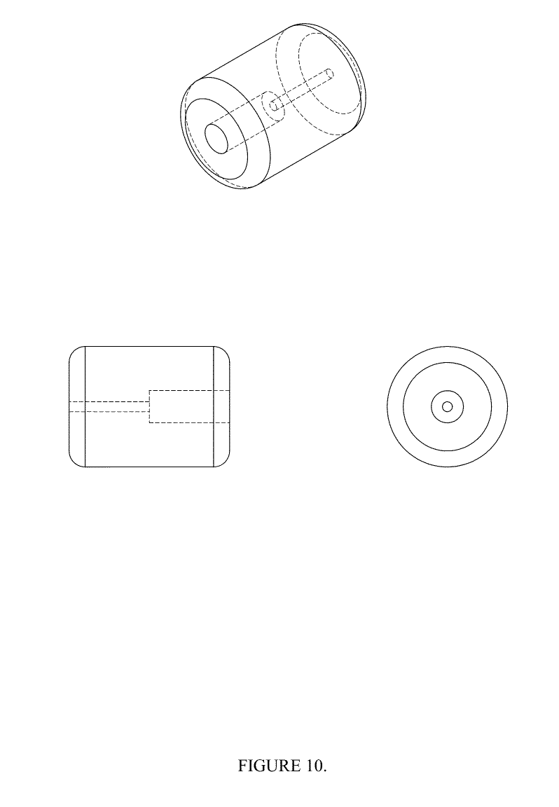

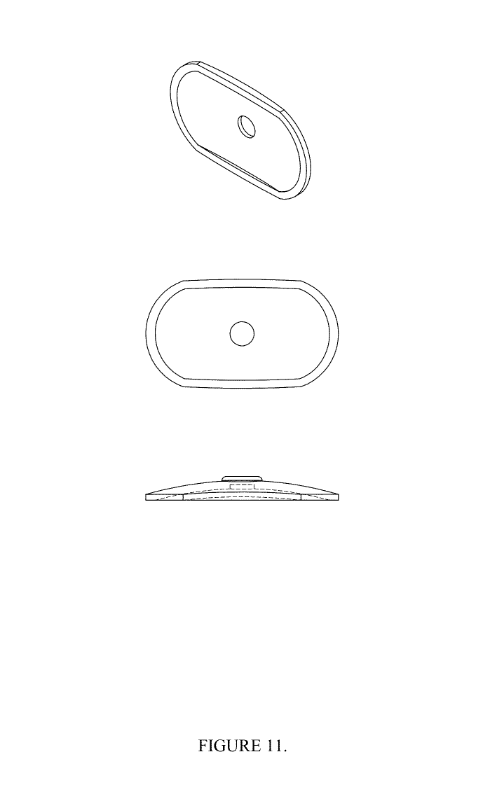

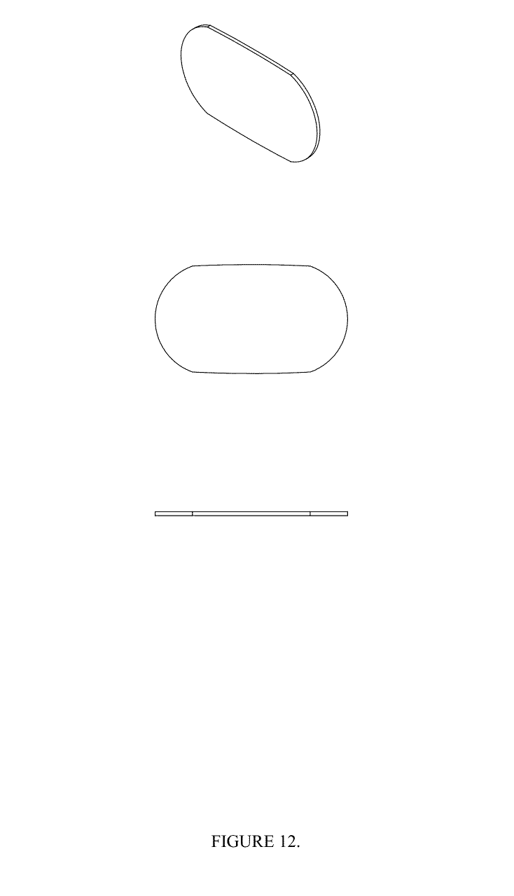

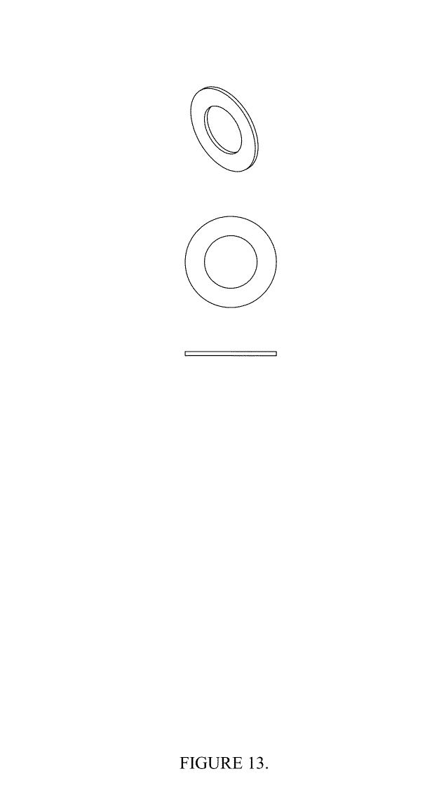

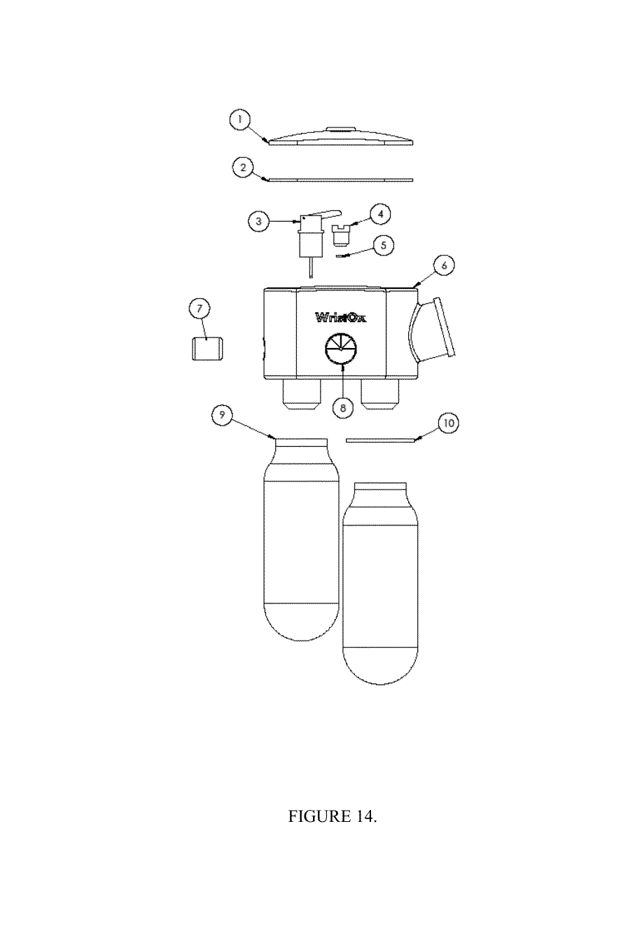

The present invention has a regulator housing (6), FIG. 3, with attachment points for the two cylinders (9). The attachment points for the cylinders are designed to provide quick attachment or removal of the cylinders. A WristOx with two full cylinders will provide a calm user with 16 breaths in standard atmospheric conditions. In the event that there is not enough available air to escape an emergency situation, alternate cylinders can be installed to extend the useable timeframe of the WristOx. The alternate cylinders can be stored in clothing pockets or in additional gear compartments for desired access during emergency situations.

Once cylinders (9) are attached to the regulator housing (6), the pressure gauge (8) will provide an accurate reading of the remaining air contained within the unit. This gauge (9) can serve as an indicator of unit readiness during normal non-emergency reviews and checks.

Inside the regulator housing (6) is a single stage regulator that will reduce the air pressure from 4,500 psi to ambient pressure. Because there is no hose leading from the tanks to the mouthpiece, the pressure reduction can be done in a single stage, reducing the mechanical parts necessary in each unit. The regulator will only supply air when demanded through the mouthpiece.

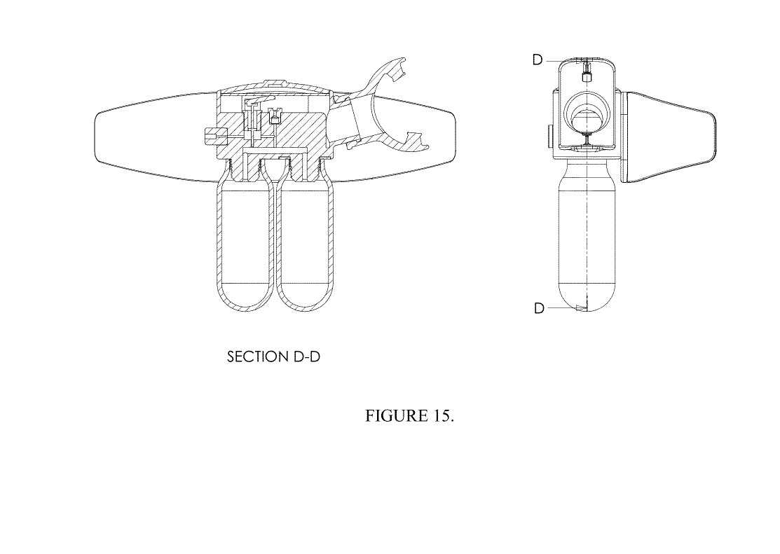

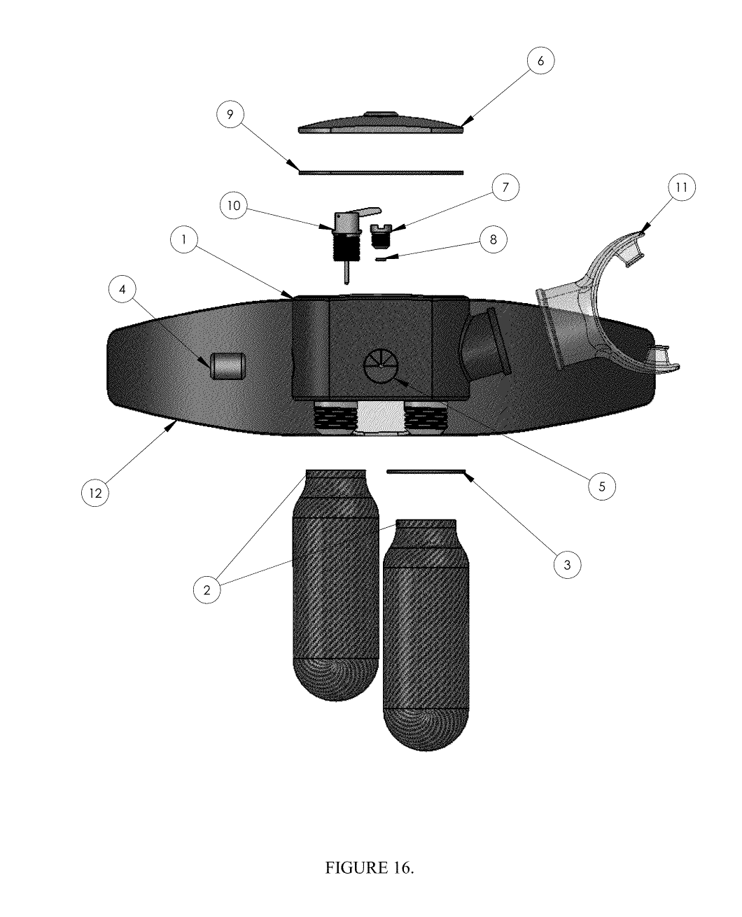

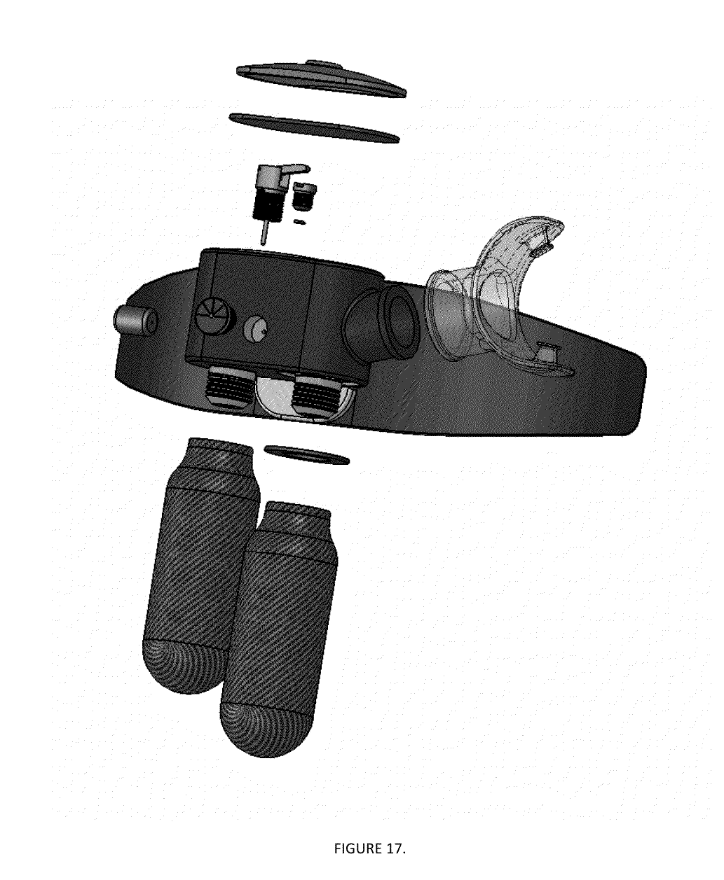

The present invention relies on the same principles as traditional regulators to allow for air to flow from the cylinders (9) to the mouthpiece (FIG. 15). Beneath the top (1) of the regulator housing (6) is a diaphragm (2). The diaphragm (2) is in direct contact with the poppet (3). Beside the poppet is the plug (4) and bust disc (5). When the user draws in air while using the mouthpiece, the diaphragm (2) flexes inward, depressing the poppet (3). The poppet (3), in turn, blocks the pathway to the check valve (7), diverting air, instead to the burst disc and plug. The air travels along this pathway and into the mouth of the user. Once the user completes the sucking action required to take a breath, the diaphragm returns to its normal position, allowing the poppet (3) to rise, thus diverting the air from the cylinders (9) back to the check valve pathway. If the user exhales into the mouthpiece, there is an exhaust disc (10) located around the perimeter of the nearest cylinder (9) that allows for the release of air.

On the opposite end of the WristOx regulator housing (6) from the mouthpiece is the check valve (7). The check valve is connected to the cylinders (9) through the poppet (3). The check valve on the present invention works in the same fashion as a traditional self-contained breathing apparatus. In addition, the check valve also functions as a means to refill empty cylinders (9) when connected to an air compressor.

The present invention has a strap along the rear of the regulator housing (6). This strap is replaceable to accommodate any desired attachment method (hook and loop, wristwatch closure, metal hook and clasp, etc.). The strap is made of rubber with a wristwatch closure. This strap allows the user the freedom to position the unit where it is most accessible. If it is fastened to the wrist or upper arm, the WristOx can be used without having to detach the unit from the user's body.

The unit will be subject to high impact speeds and shock loads, given the nature of the environments that the WristOx is most likely to be used in. As such, all of the components in the system have been designed to withstand the loads with a safety factor of two.

The present invention as shown in FIG. 1, weighs approximately 1.5 pounds fully assembled with two filled cylinders. The overall dimensions of the unit with two filled cylinders are 5.2″ in length by 4.75″ in width and 1.5″ in depth. The size and weight of the unit allow for it to be used in many applications that are currently not feasible for existing breathing apparatuses.

What is claimed:

1. A portable breathing apparatus comprising: a independent single stage regulator with the capability of selectively permitting airflow, a mouthpiece or other means with which to connect said regulator directly to a human mouth, interchangeable pressurized air cylinders connected directly to said regulator, a visible pressure gauge, and a means for attaching a strap to fasten said apparatus to a human wrist, forearm, or upper arm, all of which, when combined to form said apparatus do not interfere with normal human range of motion or activity while being worn.

2. A portable breathing apparatus as set forth in claim 1 with said pressurized air cylinders comprised of carbon fiber wrapped aluminum tanks.

3. A portable breathing apparatus as set forth in claim 1 with said regulator comprising an independent unit from the said interchangeable pressurized air cylinders, allowing for a pressurized air cylinder to be replaced while apparatus is still in use.

4. A portable breathing apparatus as set forth in claim 1 comprising attachment points for a plurality of pressurized air cylinders, only one of which must be attached for said apparatus to function.

5. A portable breathing apparatus as set forth in claim 1 comprising a method of connecting said apparatus to an external compressed air source and filling compressed air cylinders attached to apparatus.

6. A portable breathing apparatus as set forth in claim 1 comprising means for dispensing a compressed volume of gases for medicinal purposes.
